(19) United States
(12) Patent Application Publication
(10) Pub. No.: US 2004/0157930 A1
(43) Pub. Date: Aug. 12, 2004

(54) Histone deacetylase enzyme-inhibiting derivatives of hydroxamic acid as new cytokine synthesis-inhibiting anti-inflammatory drugs

(76) Inventors: Paolo Mascagni, Milano (IT); Flavio Leoni, Milano (IT); Giulia Porro, Milano (IT); Paolo Pagani, Milano (IT); Giancarlo Dona, Milano (IT); Pietro Pozzi, Milano (IT); Charles Dinarello, Milano (IT); Giamila Fantuzzi, Denver, CO (US); Britta Siegmund, Milano (IT); Leonid Reznikov, Milano (IT); Philip Bufler, Milano (IT); Soo Hyun Kim, Aurora, CO (US); Benjamin Pomeranz, Milano (IT)

Correspondence Address: NIXON & VANDERHYE, PC, 1100 N GLEBE ROAD, 8TH FLOOR, ARLINGTON, VA, 22201-4714, US

(21) Appl. No.: 10/486,152

(22) PCT Filed: Jul. 26, 2002
(86) PCT No.: PCT/EP02/08379

(30) Foreign Application Priority Data
Aug. 7, 2001 (IT) MI01A001733

(51) Int. Cl. A61K 31/19

(52) U.S. Cl. 514/575

(57) ABSTRACT

The use of derivatives of hydroxamic acid having histone deacetylase enzyme-inhibiting activity for the preparation of anti-inflammatory medicaments is disclosed.

This invention relates to the use of hydroxamic acid derivatives having histone deacetylase enzyme-inhibiting activity for the preparation of anti-inflammatory medicaments.

Some derivatives of hydroxamic acid which inhibit histone deacetylases are known. Those which have been most extensively studied are suberoylanilide hydroxamic acid (SAHA), N-hydroxy-3-[3-(hydroxyamino)-3-oxo-1-propenyl]-benzamide (CBHA) and trichostatin (TSA). Other derivatives are described in Proc. Natl. Acad. Sci. USA 95, 3003-3007, 1998; Tumori, 2001 November-December, 87 (6): S12-4; Anticancer Drugs, 2002 January, 13 (1): 1-13; Nature Rev Cancer, 2001 December, 1 (3): 194-202; Curr Opin Oncol, 2001 November 13 (6): 477-83; Cancer Chemother Pharmacol, 2001, August, 48 Suppl 1:S20-6; Cancer Chemother Pharmacol, 2001 August, 48 Suppl 1:S17-9; Haematologica, 2001 September; 86 (9): 908-17.

These compounds have mainly been studied as potential anti-tumoral drugs: trichostatin, an antifungal antibiotic isolated from Streptomyces hygroscopicus, is a potent inducer of murine erythroleukaemic cell differentiation (Cancer Res. 47, 3288-3691, 1987), while SAHA and CBHA have been studied by the Sloan Kettering Institute (WO 95/31977) as tumour cell differentiation inducing agents.

The therapeutic use of histone deacetylase inhibitors to treat tumours is described in Anticancer Res. 20, 1471-1486, 2000 and Exp.Opin.Invest. Drugs 8(10),1611-1621,1999.

It has now been found that the known derivatives of hydroxamic acid having histone deacetylase inhibiting activity, especially trichostatin and SAHA, inhibit the synthesis of pro-inflammatory cytokines, and can therefore be used to treat disorders which can be alleviated by inhibiting those cytokines. Examples of such disorders, with an inflammatory and/or autoimmune basis, include multiple sclerosis, Crohn's disease and ulcerative colitis, atherosclerosis, rheumatoid arthritis, psoriasis, spondyloarthropathies (anchilosating spondilitis, psoriatic arthritis, arthritis connected to ulcerative colitis), AIDS-related neuropathies, asthma, chronic obstructive lung diseases, bronchitis, pleuritis, acute and chronic hepatitis (either viral, bacterial or toxic), acute glomerulonephritis and, broadly speaking, all disorders with an inflammatory component

For the therapeutic uses considered, the hydroxamic acid derivatives will be administered at doses ranging between 1 and 500 mg one or more times a day, depending on the disorder concerned and the pharmacotoxicological characteristics of the compound in question, which can be administered in the form of suitable oral, parenteral or topical formulations.

The following examples illustrate the invention in greater detail.

### EXAMPLE 1

### Inhibition of Cytokine Production in Vitro

The treatment of lymphocytes with lipopolysaccharide (LPS) induces the production of various pro-inflammatory cytokines, such as TNFα, IL-1β and IFNγ (J. Biol. Chem. 1990; 265(18): 10232-10237; Science, 1998; 281:1001-1005).

The effect of SAHA and TSA has been studied by evaluating the inhibitory effect of the compound on cytokine production by peripheral blood mononuclear cells (PBMC) from healthy volunteers (2 to 6 donors), stimulated with LPS.

Samples of peripheral blood or buffy coats from healthy volunteers were used. The samples were separated by centrifugation on density gradient using Ficoll-Hypaque, and the PBMCs thus obtained were seeded in 96-well dishes (500,000 cells/well), incubated for 60 minutes with SAHA or TSA at various doses, and then stimulated with LPS from E. coli O55:B5 (10 ng/ml) for 24 hours in the presence of the compound. At the end of the treatment the pro-inflammatory cytokines TNFα, IL-1β were measured by means of an electrochemiluminescence assay (ECL) using specific commercial antibodies.

Interferon γ (IFNγ) was measured with a commercially available ELISA assay.

Cytokine IFNγ is produced by the T lymphocytes following their stimulation by pro-inflammatory cytokines, especially IL-12 and IL-18 (Dinarello C. A. and Moldawer L. L. Proinflammatory and anti-inflammatory cytokines in rheumatoid arthritis. A primer for clinicians. 2^{nd} Edition, Amgen Inc., 2000).

The effect of SAHA and TSA on IFNγ synthesis induced by stimulating PBMCs with IL-12 and IL-18 in vitro was evaluated on this basis. PBMCs were seeded in round-bottomed 96-well dishes (500,000 cells/dish), and incubated with various doses of SAHA or TSA for 60 minutes. At the end, the cells were stimulated for 48 hours in the presence of the compound by simultaneous addition of recombinant IL-12 (10 ng/ml) and recombinant IL-18 (20 ng/ml). The quantity of IFNγ produced was determined with a commercial ELISA assay.

The effect of the various doses of SAHA and TSA was measured as the percentage inhibition of production of the cytokine in question compared with untreated control cells. The concentration able to induce 50% inhibition of cell growth (IC₅₀) was determined by linear regression. The results are summarised in the table below:

| | SAHA | TSA |
|---|---|---|
| Cytokine | IC₅₀ (nM) | IC₅₀ (nM) |
| TNFα | 200 | 50 |
| IL-1β | 100 | 100 |
| IFNγ | 50 | 10 |
| IFNγ | 740 | 490 |
| (from IL-12 + IL-18) | | |

These results clearly indicate that SAHA and TSA inhibit synthesis of all the inflammatory cytokines induced by LPS with an IC₅₀ in the nanomolar range (50-200 nM).

SAHA and TSA also inhibit the synthesis of IFNγ by the T lymphocyte cells, as demonstrated by their efficacy (IC₅₀ 740 nM and 490 nM respectively) when the stimulus used was the combination of IL-12 and IL-18 specific for that cell line.

### EXAMPLE 2

### Inhibition of Cytokine Production in Vivo

Systemic administration of LPS to laboratory animals is known to induce rapid, massive production of pro-inflammatory cytokines (Immunopharmacol. 1992; 14(6): 1045-1050).

Female BALB/c mice (20-22 grams) were treated orally with SAHA at the various doses indicated, then treated after 60 minutes with LPS from E. Coli O55:B5 (30 mg/Kg intraperitoneally). 90 minutes after the endotoxin administration, blood samples were taken from all the treated animals (10 animals/group), and the cytokines were measured with commercial ELISA assays.

The results are set out in the table below, and expressed as the percentage inhibition of production of the cytokine in question:

| | % | Inhibition | Inhibition |
|---|---|---|---|
| | inhibition of | of | of |
| TREATMENT | TNFα | IL-1β | IL-6 |
| SAHA | | | |
| 0.1 mg/Kg | 40 | 13 | 10 |
| 1 mg/Kg | 53 | 15 | 3 |
| 10 mg/Kg | 67 | 35 | 7 |
| 25 mg/Kg | 68 | 37 | 25 |
| 50 mg/Kg | not done | 51 | 29 |

The above results indicate that SAHA is active orally and able to inhibit, to a dose-dependent extent, pro-inflammatory cytokine synthesis induced in vivo in the mouse by administering endotoxin, thus confirming the results obtained in vitro.

### EXAMPLE 3

### Con A-Induced Liver Injury

C57B16 mice were injected i.p. with either water vehicle or SAHA and after 1 h were injected i.v. with Con A as described in Proc. Natl. Acad. Sci. USA, (2000), 97, 2367-2372. After 24 h, serum amino-alanine transferase was measured.

Intravenous injection of Con A results in hepatic cell death within 12-24 h with markedly elevated serum levels of hepatic enzymes such as alanine amino transaminase (ALT). In mice pretreated with a single dose of SAHA (50 mg/kg) given i.p. 1 h before Con A, the 24-h level of serum ALT (mean±SE) was 8.144±2.091 units/liter compared with 15.190±2.580 in vehicle-treated mice (n=6 per group).

### EXAMPLE 4

### Nitric Oxide Production from Mouse Peritoneal Macrophages

C57BLy6 mice were injected i.p. with 1 ml of sterile thioglycolate broth and killed after 5 days, and macrophages were isolated using instillation of 10 ml of ice-cold PBS into the peritoneal cavity. The cells were centrifuged (350×g) and 3 ml of erythrocyte lysing reagent (PharMingen) was added for 10 min.

Seven milliliters of DMEM containing 5% FCS was added and the cells were centrifuged at 4° C. The cells were resuspended in DMEM at 1 million per ml and 0.5 ml were added to wells of a 48-well plate. SAHA was added for 60 min at 37° C. and then stimulated with the combination of TNF-α plus IFN-γ. After 24 h, NO levels in the supernatant were determined using the Griess reagent as described in Am.J.Physiol.Cell Physiol. (2001), 280, C441-C450.

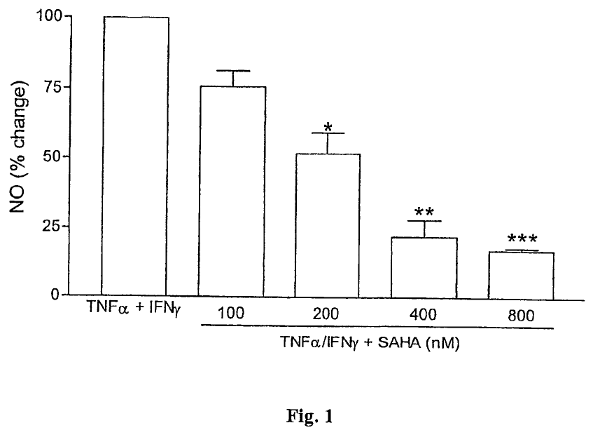

As shown in FIG. 1, SAHA inhibited NO production; at 200 nM, there was a 50% reduction (P, 0.05). Further reductions of 80 and 85% were observed at 400 and 800 nM, respectively.

### EXAMPLE 5

### Inhibition of IL-12 Production by Cultured Monocytes

Venous blood was obtained from consenting adults and separated over Ficoll-Hypaque. The PBMC fraction was washed and adjusted to five million cells per ml. Five hundred microliters was aliquoted into each well of 24-well flat-bottom plates, 100 ml of SAHA was added, and the plates were incubated for 1 h at 37° C. The cells were stimulated with LPS, soluble OKT3, or cytokines, and after 24 or 48 h At 37° C. the supernatant was removed and frozen for cytokine assays. Monocytes were isolated by centrifugation over Percoll, washed, suspended in RPMI with 10% FCS, and aliquoted at 2 million cells per ml in Petriperm Teflon-coated culture dishes (Sigma). The ELISA for human IL-12 (p70) was purchased from Endogen (Woburn, Mass.).

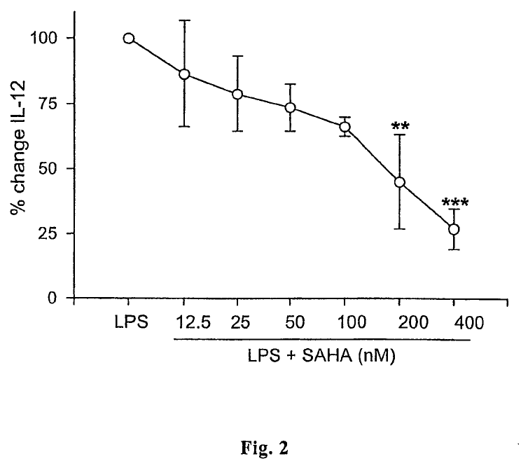

As shown in FIG. 2, there was a dose-dependent reduction in LPS/IFN-γ-induced IL-12 production in nonadherent human monocytes. At 200 nM, the reduction was 55% (P, 0.01) and at 86% at 400 nM (P, 0.001).

### EXAMPLE 6

### Dextran-induced Colitis

Female, 8 week-old C57BL/6 mice (The Jackson Laboratories, Bar Habor, Me.) weighing 20-22 g were used in this study. The animals were housed in rooms at a controlled temperature and a 12 h day/night rhythm. They were fed standard mice chow pellets ad libitum, had free access to tap water supplied in bottles, and were acclimatized to the conditions at least seven days before they were used in experiments. Mice were killed by cervical dislocation under isoflurane anesthesia (Fort Dodge, Iowa City, Iowa).

Mice were fed 3.5% dextran sulfate sodium (DSS; molecular weight 40 kDa; ICN, Aurora, Ohio) dissolved in sterile, distilled water ad libitum from day one to five followed by a five day observation period. SAHA was administered once daily orally (p. o.) in a total volume of 200 μl and a concentration of 10 mg/kg body weight (BW) throughout the experiment (day 1 to 10). Control mice had free access to water and received either SAHA (10 mg/kg BW) or water p. o. once daily for 10 days.

Body weights, occult blood or the presence of gross blood per rectum, and stool consistency were determined daily. Two investigators blinded to the protocol assessed the clinical score (table 1). Weight loss <1% compared to day 1 was counted as 0 points, weight loss of 1 to <5% as 1 point, 5 to <9.9% as 2 points, 10 to 20% as 3 points and more than 20% as 4 points. For stool consistency, 0 points were given for well-formed pellets (formed), 2 points for pasty and semi-formed stools which did not stick to the anus (soft), and 4 points for liquid stoops that did stick to the anus (diarrhea). Bleeding was scored 0 points for no blood in hemoccult, 2 points for positive hemoccult, and 4 points for gross bleeding. These scores (body weight, stool consistency, rectal bleeding) were added and divided by 3 resulting in a total clinical score ranging from 0 (healthy) to 4 (maximal activity of colitis).

Post mortem (on day 10), the entire colon was removed from the caecum to the anus and the colon length was measured as an indirect marker of inflammation. Colon length has been shown to be a reliable parameter in this model as DSS-induced colitis is associated with colon shortening as described previously [Gastroenterology,1990, 98,:694:J. Pharmacol.Exp.Ther., 2001, 296:99-105].

From the results, reported in the following Tables 1-6, it is evident that SAHA effectively counteracts dextran-induced colitis, a valid and established model of inflammatory bowel diseases in humans.

TABLE 1
Clinical activity score [Lab. Invest., 1993, 69: 238-249].

| Score | | Stool | |
|---|---|---|---|
| points | Weight loss | consistency | Rectal bleeding |
| 0 | 0% | Formed | Negative hemoccult |
| 1 | (>0%) <5% | | |
| 2 | 5-9.9% | Soft | Positive hemoccult |
| 3 | 10-20% | | |
| 4 | >20% | Diarrhea | Macroscopic bleeding |

TABLE 2
Weight

| | Days | | | | | | | | | |
|---|---|---|---|---|---|---|---|---|---|---|
| Group | 1 | 2 | 3 | 4 | 5 | 6 | 7 | 8 | 9 | 10 |
| DSS + SAHA | 19.1 ± 0.2 | 18.8 ± 0.2 | 19.1 ± 0.3 | 18.4 ± 0.4 | 18.4 ± 0.4 | 18.7 ± 0.3 | 18.0 ± 0.4 | 16.9 ± 0.3 | 16.6 ± 0.4 | 16.6 ± 0.4 |
| DSS + water | 18.7 ± 0.5 | 19.0 ± 0.5 | 19.4 ± 0.5 | 18.9 ± 0.5 | 18.9 ± 0.5 | 18.8 ± 0.6 | 18.8 ± 0.6 | 16.6 ± 0.5 | 16.8 ± 0.5 | 16.8 ± 0.5 |
| SAHA | 18.9 ± 0.3 | 18.8 ± 0.4 | 18.9 ± 0.2 | 18.9 ± 0.3 | 19.0 ± 0.1 | 19.0 ± 0.3 | 18.9 ± 0.1 | 19.0 ± 0.3 | 19.1 ± 0.1 | 19.1 ± 0.2 |
| Water | 19.0 ± 0.2 | 19.0 ± 0.3 | 18.9 ± 0.1 | 19.0 ± 0.2 | 19.0 ± 0.4 | 19.0 ± 0.2 | 19.1 ± 0.2 | 19.1 ± 0.1 | 19.1 ± 0.3 | 19.1 ± 0.1 |

TABLE 3
Stool consistency as score

| | Days | | | | | | | | | |
|---|---|---|---|---|---|---|---|---|---|---|
| Group | 1 | 2 | 3 | 4 | 5 | 6 | 7 | 8 | 9 | 10 |
| DSS + water | 0.0 ± 0.0 | 0.0 ± 0.0 | 0.0 ± 0.0 | 0.4 ± 0.4 | 1.2 ± 0.4 | 2.0 ± 0.0 | 2.0 ± 0.0 | 2.0 ± 0.0 | 1.6 ± 0.4 | 1.6 ± 0.4 |
| DSS + SAHA | 0.0 ± 0.0 | 0.0 ± 0.0 | 0.0 ± 0.0 | 0.0 ± 0.0 | 0.8 ± 0.4 | 0.8 ± 0.4 | 2.0 ± 0.0 | 2.0 ± 0.0 | 1.2 ± 0.4 | 1.2 ± 0.4 |
| SAHA | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |
| Water | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |

TABLE 4
Bleeding

| | Days | | | | | | | | | |
|---|---|---|---|---|---|---|---|---|---|---|
| Group | 1 | 2 | 3 | 4 | 5 | 6 | 7 | 8 | 9 | 10 |
| DSS + water | 0.0 ± 0.0 | 0.0 ± 0.0 | 0.0 ± 0.0 | 0.8 ± 0.4 | 1.6 ± 0.4 | 2.8 ± 0.4 | 2.0 ± 0.0 | 2.0 ± 0.0 | 2.0 ± 0.0 | 1.6 ± 0.4 |
| DSS + SAHA | 0.0 ± 0.0 | 0.0 ± 0.0 | 0.0 ± 0.0 | 0.0 ± 0.0 | 1.2 ± 0.4 | 2.4 ± 0.4 | 2.0 ± 0.0 | 1.6 ± 0.4 | 1.2 ± 0.4 | 0.8 ± 0.4 |
| SAHA | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |
| Water | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |

TABLE 5
Complete clinical score

| | Days | | | | | | | | | |
|---|---|---|---|---|---|---|---|---|---|---|
| Group | 1 | 2 | 3 | 4 | 5 | 6 | 7 | 8 | 9 | 10 |
| DSS + water | 0.0 ± 0.0 | 0.3 ± 0.1 | 0.2 ± 0.1 | 0.8 ± 0.1 | 1.1 ± 0.2 | 2.1 ± 0.1 | 2.0 ± 0.1 | 2.1 ± 0.1 | 2.0 ± 0.1 | 1.9 ± 0.2 |
| DSS + SAHA | 0.0 ± 0.0 | 0.0 ± 0.0 | 0.1 ± 0.1 | 0.2 ± 0.1 | 0.7 + 0.3 | 1.3 ± 0.3 | 1.5 ± 0.1 | 1.9 ± 0.1 | 1.5 ± 0.4 | 1.3 ± 0.4 |
| SAHA | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.1 | 0.0 ± 0.0 |
| Water | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |

TABLE 6
Colon length

| Group | Colon length (cm) |
|---|---|
| DSS + water | 7.6 ± 0.2 |
| DSS + SAHA | 9.2 ± 0.2 |
| SAHA | 10.1 ± 0.2 |
| Water | 10.5 ± 0.3 |

### EXAMPLE 7

A series of hydroxamic acid derivatives described in EP 901465 were subjected to the histone deacetylase (HDAC) and TNFα inhibition tests in accordance with the methods described by Lechner et al., Biochim Biophys. Acta 1996, 1296, 181-188 and Moreira A. L. et al., J. Exp. Med. 1993, 177-1657-1680 respectively.

The results, set out in the following table, show that a linear correlation exists between the ability of these compounds to inhibit the synthesis of TNFa and their inhibition of HDAC activity.

| | 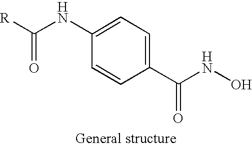 | | | | |
|---|---|---|---|---|---|
| | General Structure | HDAC | | TNF | |
| | R | IC50 nM | Potency | IC50 nM | Potency |
| 1 | 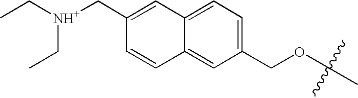 | 20.0 | 100.00 | 7.0 | 100.00 |
| 2 | 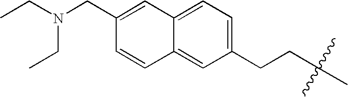 | 62.0 | 32.26 | 10.2 | 68.75 |
| 3 | 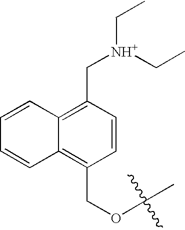 | 65.0 | 30.77 | 10.3 | 68.02 |
| 4 | 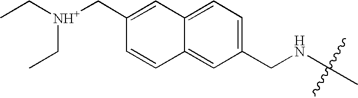 | 76.0 | 25.64 | 11.2 | 62.68 |
| 5 | 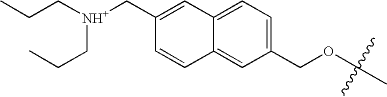 | 46.7 | 42.86 | 12.7 | 55.28 |
| 6 | 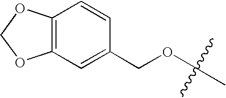 | 80.0 | 25.00 | 50.0 | 14.00 |
| 7 | 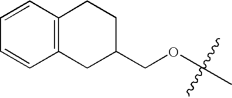 | 91.0 | 21.98 | 65.5 | 10.68 |
| 8 | 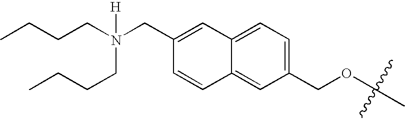 | 133.3 | 15.00 | 67.6 | 10.32 |
| 9 | 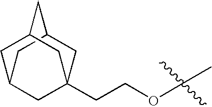 | 600.0 | 3.33 | 159.1 | 4.40 |
| 10 | 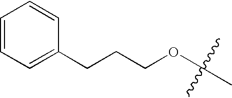 | 105.0 | 19.05 | 159.1 | 4.40 |
| 11 | 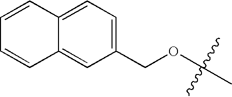 | 8.1 | 246.91 | 159.1 | 4.40 |
| 12 | 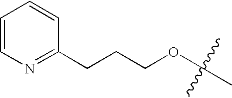 | 260.0 | 7.69 | 230.0 | 3.04 |
| 13 | 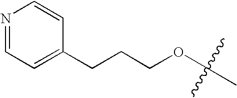 | 260.0 | 7.69 | 270.0 | 2.59 |
| 14 | 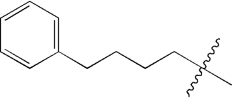 | 86.7 | 23.08 | 300.0 | 2.33 |
| 15 | 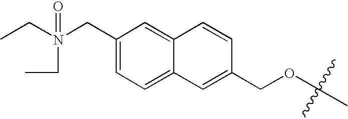 | 206.7 | 9.68 | 1000.0 | 0.70 |

1. The use of hydroxamic acid derivatives having histone deacetylase inhibiting activity for the preparation of anti-inflammatory medicaments.

2. Use as claimed in claim 1, wherein the derivatives are selected from suberoylanilide hydroxamic acid (SAHA), N-hydroxy-3-[3-(hydroxyamino)-3-oxo-1-propenyl]-benzamide (CBHA) and trichostatin (TSA).

3. Use as claimed in claim 2, wherein the derivatives are selected from suberoylanilide hydroxamic acid (SAHA) and trichostatin (TSA).

4. Use as claimed in claim 1 or 2 for the preparation of medicaments for the treatment of multiple sclerosis, Crohn's disease and ulcerative colitis, atherosclerosis, rheumatoid arthritis, psoriasis, spondyloarthropathies (anchilosating spondilitis, psoriatic arthritis, arthritis connected to ulcerative colitis), AIDS-related neuropathies, asthma, chronic obstructive lung diseases, bronchitis, pleuritis, acute and chronic hepatitis (either viral, bacterial or toxic), acute glomerulonephritis.
